(19) United States
(12) Patent Application Publication
(10) Pub. No.: US 2002/0151037 A1
(43) Pub. Date: Oct. 17, 2002

(54) Microbiological controlled mycoculture nutrient substrates

(76) Inventors: Theo van der Sterren, Horst (NL); Ger Hendriks, Ottersum (NL); Geert Lemmers, Valkenswaard (NL); Mark Peter Wach, Allison Park, PA (US)

Correspondence Address: Woodcock Washburn Kurtz, Mackiewicz & Norris LLP, 46th Floor, One Liberty Place, Philadelphia, PA, 19103, US

(21) Appl. No.: 09/779,067

(22) Filed: Feb. 8, 2001

(51) Int. Cl. C12N 1/14; C12N 1/16; C12N 1/18

(52) U.S. Cl. 435/254.1; 435/256.8

(57) ABSTRACT

The present invention is directed to the controlled preparation of mycoculture nutrient substrates and in particular, to methods of composting which include the enzymatic digestion of substrate via colonization with microbial cultures or enzymes. The method of the present invention also include the colonization of substrate with microbial cultures for conferring increased microbial nitrogen content to such substrate. The present invention is further directed to mushroom substrate produced via the methods described herein.

### FIELD OF THE INVENTION

The present invention relates to the field of preparation of nutrient substrates for mycoculture and, more particularly, to methods of composting such substrates which include microbial cultures or enzymes for the enzymatic digestion of and conferrance of increased nitrogen content to such substrates, as well as mushroom substrates produced via such methods.

### BACKGROUND OF THE INVENTION

Commercially cultivated mushrooms such as Agaricus bisporus are generally grown on a nutritional compost prepared from wheat straw, hay, horse manure, chicken manure, broiler chicken manure, gypsum or other organic material. Such compost is commonly referred to as substrate, and its preparation referred to as composting. The process of composting is governed by the basic principles of heat and mass transfer and the biological constraints of living organisms. Keener et al., Transactions of the ASAE: American Society of Agricultural Engineers 39(5): 1839-1845 (1996). Some researchers have been able to establish operational parameters for the limited success of certain processes.

Typical processing of mushroom substrate includes a largely uncontrolled, often outdoor, Phase I, and an environmentally controlled Phase II. Phase I typically includes mixing and moistening of organic materials and a period characterized by an uncontrolled temperature rise. Phase II starts with a pasteurization period and continues with a conditioning period typically until volatile ammonia has been cleared from the process air. After Phase II, the substrate is ready for growth of mushroom mycelium. Typically, current composting technology involves premixing raw materials either outdoors via a large “mixing line” or using large tractors to form the substrate. The substrate is then placed in an enclosed space (tunnel) with limited environmental control for a period of 5-7 days. The substrate is subsequently transferred to a bulk pasteurization chamber where it is occasionally inoculated with previously pasteurized substrate material. The substrate is typically pasteurized within 1-2 days. The substrate is then subjected to an extended conditioning period of 4-8 days, the period characterized by high levels of ammonia production. Finally, the substrate is cooled to room temperature (˜25° C.) and mushroom spawn added.

Important parameters of ready-to-spawn substrate can include the absence of volatile ammonia; the absence of actively growing thermophilic fungi including Scytalidium thermophilum; and the amount and type of nitrogenous components. Straatsma et al., Bioresource Technology 72: 67-74 (2000). In addition, the substrate should have a high weight per unit volume to fill the cultivation area with a high amount of substrate, but a relatively rigid texture must remain for gas and water exchange. However, it is not possible to directly establish these parameters. Although moisture, nitrogen content and pH can be adjusted at the start of Phase I, the values are affected during processing. In addition, activity during Phase I is determined largely by microbial response to physical factors such as temperature and O₂ limitations. Miller et al., Australian Journal of Experimental Agriculture 29: 741-50 (1989).

Various approaches to mushroom substrate production have been reviewed by Fermor et al., Compost as a substrate and its preparation, in The Biology and Technology of the Cultivated Mushroom pp. 81-110 (P. B. Flegg et al. eds. 1985). Improving traditional composting processes has been difficult because precise ecological control has not been possible during Phase I, while activity analysis has been frustrated by temporal and spatial variability in composting stacks. The development of alternative environmentally controlled composting (ECC) processes has been pursued as a means of improving commercial practice. Some investigators have produced mushroom substrate under conditions of much greater environmental control within enclosed systems. Harper et al., Australian Journal of Experimental Agriculture 32: 657-667 (1992). Control strategies have varied, however, with different approaches arising due to uncertainty in the definition of a good substrate for mushroom production.

New processing variations can be difficult to implement because of an incomplete knowledge of the physical factors influencing composting processes. Harper et al., supra. Mycological substrate is an ecosystem that responds strongly to changes in physical factors that select for microbial activity. Accordingly, different processing scenarios can lead to different outcomes. Conversely, composting activity can profoundly affect physical factors such as temperature, O₂ concentration and moisture content. More information regarding the substrate ecosystem is required for the better design of traditional composting processes.

Certain important factors have been identified. Temperature has been identified as a primary factor in controlling the composting process. Harper et al., supra. The management of metabolically released heat has been the means of controlling temperature. Amount of O₂ is also an important factor in the process of composting. Other physical factors related to mass, gas transfer and moisture content also affect the process. Investigators have pursued a variety of mushroom composting trials in which ECC processes are used to gain control over temperature, concentration, mass change and other physical factors. Harper et al., supra.; Miller et al., Australian Journal of Experimental Agriculture 30: 287-96 (1990).

No matter the particular composting process, the digestion or degradation of solid organic materials by microorganisms is an important step. The breakdown of solid organic materials can be accomplished either aerobically or anaerobically. Phase I is important in this respect, providing for the digestion of substrate materials, such as straw, so as to allow for an optimal amount of substrate being filled into the cropping rooms and for an optimal conversion of substrate into mushroom fruit body biomass. While mushroom yield may be 20% lower when Phase I is not applied, it has been reported that the level of degradation has no relation to the yield of the mushroom. Gerrits et al., Champignoncultur 41: 179-83 (1997). Rather, degradation is relevant for the quantity of substrate produced and, since the ratio of water and of dry matter is not constant, degradation plays a role in the final dry matter contents of the substrate. Straatsma et al., Bioresource Technology 72: 67-74 (2000).

Traditional composting procedures are subject to a variety of other problems in addition to the problem of digestion. For example, ammonia and foul smelling compounds are generally emitted during Phase I, causing environmental problems. In the Netherlands and other countries it is no longer permitted to perform Phase I in the open air due to the emission of ammonia and stench. Gerrits et al., Phase I compost in tunnels for the production of Agaricus bisporus compost with special reference to the importance of water, in Science and Cultivation of Edible Fungi 203-211 (T. J. Elliott ed. 1995). Other problems related to Phase I include pollution, variations in substrate quality as well as the inefficient utilization and difficulty in handling raw materials. Harper et al., Australian Journal of Experimental Agriculture 32: 657-67 (1992). Various problems are also associated with Phase II. In particular, Phase II is characterized by the utilization of carbohydrates and the disappearance of ammonia. It is, thus, attended by the disappearance of available nutrients that are no longer available for mushroom growth.

There is a need for improved composting processes and substrate. In particular, there is a need for methods which improve the digestion and nutritional content of substrate. It is also desired to improve the kinds of nitrogenous species extant in prepared compost to increase the levels of microbial protein at the expense of other nitrogenous materials, e.g. urea. There is also a need for substrate having improved nutritional content for mushroom growth. The present invention is directed to these, as well as other, important ends.

### SUMMARY OF THE INVENTION

The present invention provides methods for the production of mushroom substrate which include the enzymatic digestion of substrate via colonization with exogenously-derived microbial cultures or enzymes under controlled conditions.

In related aspects, the present invention provides mushroom substrate produced by the methods described herein.

### BRIEF DESCRIPTION OF THE DRAWINGS

FIG. 1 is flow chart diagram illustrating the enzymatic digestion of substrate, in accordance with certain preferred embodiments of the present invention.

FIG. 2 is a flow chart diagram illustrating the increase in substrate nitrogen content, in accordance with certain preferred embodiments of the present invention.

The present invention is based in part on the discovery that traditional methods of composting can be improved via the enzymatic digestion of substrate. It has now been discovered that substrate can be enzymatically digested with exogenously-derived microbial cultures or enzymes so as to reduce the cost and environmental impact of composting while improving the resulting product. It is now possible to provide not only substantial savings on organic raw materials, but also to reduce or prevent ammonia evolution and offensive odor. The present invention further provides for increasing the available microbial nitrogen content of substrate. Mushroom substrate is thus of a higher quality and can result in improved mushroom growth. The present invention provides significant advantages over traditional composting.

For purposes of the present invention the term “composting” refers to the production of substrate or compost suitable for the cultivation of mushrooms.

The term “digest” is understood to mean to soften, decompose or break down through biological processes. It is to be understood that the terms “digest” and “degrade” may be used interchangeably. The phrase “enzymatically digest means to perform such softening, decomposition or breakdown through use of enzymes.

The term “microbial culture” refers to a culture of any microorganism suitable for use in accordance with the present invention, including the ability to provide the same in sufficient quantity to materially improve the composting process.

It is to be understood that for purposes of this invention, the terms “substrate” and “compost” may be used interchangeably.

All references herein are hereby incorporated by reference.

Commercial mushroom growing requires a substrate suitable for providing a food source to growing mushrooms. Suitable substrate generally includes organic materials such as wheat straw, hay, rice, barley, oats, bagasse and a variety of other organic materials. Wheat straw is often the primary ingredient for the preparation of substrate for the commercial production of button mushrooms, for instance Agaricus bisporus. Such materials generally contain nitrogen, lignin and carbohydrates including cellulose and hemicellulose. However, before substrate is suitable for mushroom production, it must undergo a process wherein carbohydrates are digested or degraded so as to produce a suitable biomass.

The present invention provides for methods of composting which include the enzymatic digestion of substrate. Through the addition of microbial cultures and enzymes, substrate is enzymatically digested so as to produce a biomass suitable for mushroom production. Such enzymatic digestion can be bio-efficient in that organic raw materials are better utilized. For example, enzymatic digestion can reduce or inhibit the production of ammonia associated with traditional composting methods. Enzymatic digestion can also provide for the production of an increased quantity of substrate suitable for mushroom growth from the same amount of material. Enzymatic digestion can thus contribute significantly to the reduction of costs associated with the production of substrate. Costs may also be reduced due to the reduced investment and labor required. Production costs may be reduced by the elimination of ammonia scrubbers as required by traditional methods of composting. In addition, enzymatic digestion can provide for substrate of higher and more consistent quality, thus providing for mushroom yields that are larger and of a higher quality.

The present invention provides for improved methods of composting as well as substrate. Those skilled in the art will understand that the substrate of the present invention may comprise a variety of organic materials. However, it is preferred that such materials are capable of providing a source of nitrogen. It is also preferred that such materials provide a source of carbohydrates. Those skilled in the art will understand that organic materials suitable for use in accordance with the present invention include, by way of example only, wheat straw, hay, soybean meal, hay, lucerne meal, rapeseed meal, cottonseed meal, cotton seed hulls grape pomace, nut meal, feather meal & blood meal, rice, barley & oat straw as well as a variety of other organic materials. However, for certain preferred embodiments, it is preferred that wheat straw be a primary component of the substrate of the present invention.

It has now been discovered to be possible to “capture” nitrogen in microbial biomass in the form of mircrobial proteins. These nitrogenous materials are available to mushrooms grown in the compost and are not lost in composting. Evolution of ammonia is greatly reduced while the compost is benefitted. The transformation of ureas, plant proteins, and microbial proteins and biomass is both highly efficient and highly desirable in accordance with this invention.

The methods of the present invention include the digestion of organic components via the colonization of substrate by microbial cultures, enzymes or mixtures thereof. Those skilled in the art will understand that a variety of microbial cultures or enzymes are suitable for enzymatic digestion in accordance with the present invention. Exemplary microbial cultures suitable for enzymatic digestion in accordance with the invention are those which effectively transform the complex nitrogenous species of the rough organic constituents of the compost into microbial protein. Exemplary among these are cultures of Bacillus, especially Bacillus subtilis, Pseudomonas spp, Members of the Actinomycetes family, as well as species of Humicola, Scytalidium and others. Other particularly useful genera of microbial species include Micrococcus, Klebsiella, Micromonospora, Nocardia, Chaetomium, Melanocarpus, Paecilomyces, Thermoascus, Rhizomucor, and mixtures thereof. Enzymes suitable for enzymatic digestion include, by way of example only, cellulase, hemicellulose, laccase, proteinase, lipase and lignin peroxidase and manganese peroxides.

It will be appreciated that it is preferred to provide essentially pure cultures of the desired microbial species in order to minimize undesired pathways and to maximize effective digestion. Thus, stock cultures are preferably maintained for substrate inoculation and such maintenance is a matter of routine for persons skilled in the art. Mixtures of these cultures may also be useful. Perforce, such cultures must be capable of commercially acceptable production and delivery to compost mixtures in quantities and concentrations effective to improve the compost.

Those skilled in the art will understand that certain physical factors may influence enzymatic digestion in accordance with certain aspects of the present invention. For example, as described in Table 1 below, a variety of temperatures and time periods are suitable for the enzymatic digestion in accordance with the present invention. Those skilled in the art will further understand that moisture content, CO₂, O₂, relative humidity (“RH”) and air velocity may influence the enzymatic digestion in accordance with the present invention.

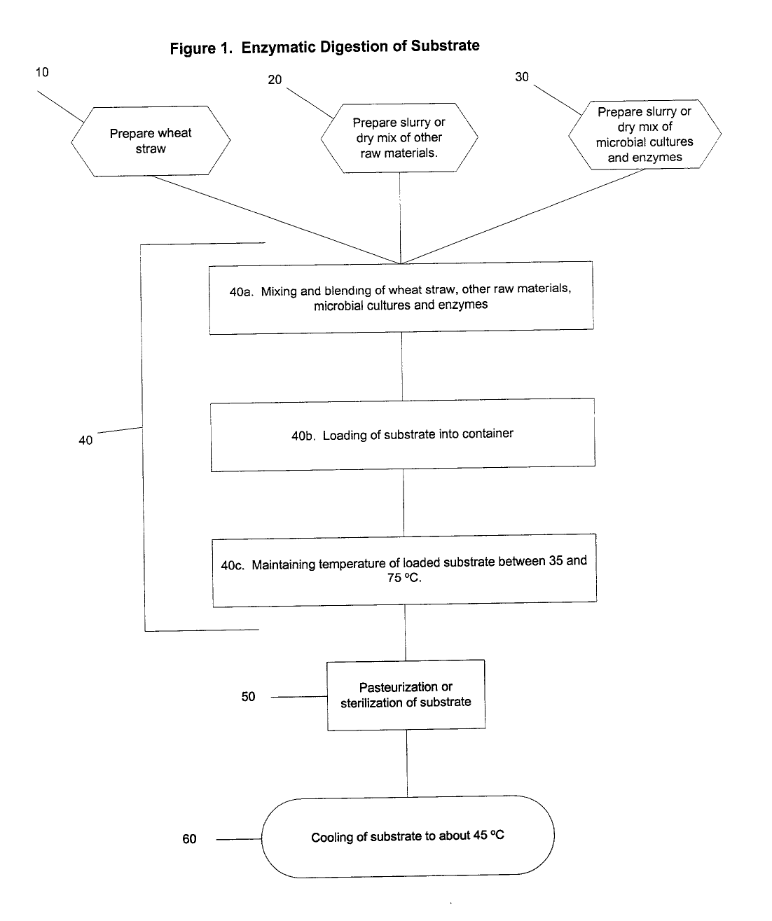

FIG. 1 provides a flow chart diagram illustrating the enzymatic digestion of substrate in accordance with preferred embodiments of the present invention. Wheat straw is prepared as an exemplary ingredient of the substrate 10. It is preferred that the wheat straw be cut into pieces of similar length so as to provide a good structure for the end product substrate. A slurry or dry mix of other suitable substrate materials containing nitrogen is prepared 20. These nitrogen containing materials can include, by way of example, soybean meal, hay, cottonseed meal, lucerne and rapeseed meal, or other materials containing different forms of organic nitrogen. Another slurry or dry mix of microbial cultures, enzymes or mixture thereof can be prepared 30. It is preferred that the slurry or dry mix includes cultures of actinomycetes and bacteria as well as enzymes.

The substrate is subjected to an actual digestion or degradation phase. The wheat straw, slurry or dry mix of other materials as well as the slurry or dry mix of microbial culture, enzyme or mixture thereof are mixed and blended 40. The mixing and blending may occur by hand, tunnel, container or a combination thereof. It is preferred mixing and blending occurs in a rotatable drum that is capable of maintaining controlled temperatures. Preferably the temperature is maintained at 45-60° C., and adjusted as necessary to optimize growth of preferred organisms, speed of rotation, O₂ content and the like. The mixed and blended substrate is loaded into a container fitted with an aeration system, 40b. It is preferred that the moisture content of the substrate is about 55 to 75% at the time of loading. It is also preferred that the substrate has a pH between about 7 to 8 at the time of loading. The temperature of the loaded substrate is maintained between about 35° and 75 ° C., 40c. It is preferred that the container be capable of maintaining temperature via aeration and cooling as necessary. Preferably CO, O_{2,} RH and air velocity are also measured and controlled. It is to be understood that during the digestion or degradation phase, 40 it is preferred that the moisture content of the substrate be continuously increased relative to the ability of the substrate to retain absorbed water. It is also preferred that the digestion or degradation phase 40 last approximately 1½-3, preferably about 2 days.

The substrate is preferably subjected to pasteurization or sterilization, 50. The substrate may be pasteurized or sterilized so as to ensure that it is essentially free of undesired microorganisms, including microorganisms that might be harmful to mushroom growth. This pasteurization or sterilization phase is characterized by an increase in temperature of the substrate due to microbial metabolism. This increase in temperature may be supplemented by the addition of steam or other heat source. Table 1 indicates exemplary combinations of time and temperature suitable for pasteurizing or sterilizing the substrate.

TABLE 1
Pasteurizing or Sterilizing Time and Temperature

| | | | |
|---|---|---|---|
| Combination 1 | 12 | hours | 84° C. |
| Combination 2 | 6 | hours | 90° C. |
| Combination 3 | 2 | hours | 100° C. |
| Combination 4 | 10 | minutes | 170° C. |

It is presently believed that such increased temperature liberates an essential microbial food source, i. e., carbohydrates, from the substrate materials. Those skilled in the art will understand that special attention must be paid to the structure of any container when employing the temperature of Combination 4 of Table 1. Finally, the substrate may be cooled to about 45° C., 60.

The present invention also provides for methods of improving nitrogen content of substrate via colonization with certain microbial cultures. In particular, substrate can be colonized with microbial cultures so as to increase the nitrogen content of the substrate relative to the uncolonized substrate. However, it is also believed that colonization of substrate with certain microbial cultures can result in the further digestion of the substrate into nutrients for mushroom growth. It is preferred that the microbial cultures are exogenously-derived relative to the substrate.

The present invention provides for increasing the relative nitrogen content of the substrate. In particular, the present invention provides for increasing the nitrogen content of the substrate relative to substrate not colonized with microbial culture. It has been demonstrated that the increase in microbial nitrogen content of the substrate can be at least about 25% based upon the nitrogen content of the uncolonized substrate. It has been demonstrated that the increase in nitrogen content of the substrate can be at least about 50% based upon the nitrogen content of the uncolonized substrate since, as the dry weight shrinks the apparent N₂ goes up. It has further been demonstrated that the increase in nitrogen content of the substrate can be at least about 25% based upon the nitrogen content of the uncolonized substrate. Those skilled in the art will understood that any increase in nitrogen content may be affected by the initial nitrogen content of the materials making up the substrate and the length of the composting process.

The present invention includes substrate having increased levels of nitrogen content. For example, it has been demonstrated that the nitrogen content of the substrate of the invention can be at least about 2.2% based upon the dry weight of substrate. It has also been demonstrate that the nitrogen content of the substrate of the invention can be about 2.2 to 2.5% based upon the dry weight of the substrate. The present invention also provides for substrate capable of providing improved mushroom growth. With reference to Tables 2 and 3, the substrate of the present invention can provide for improved mushroom yield in terms of kg harvested mushrooms/m² of substrate or kg mushrooms per ton of substrate relative to traditional substrate.

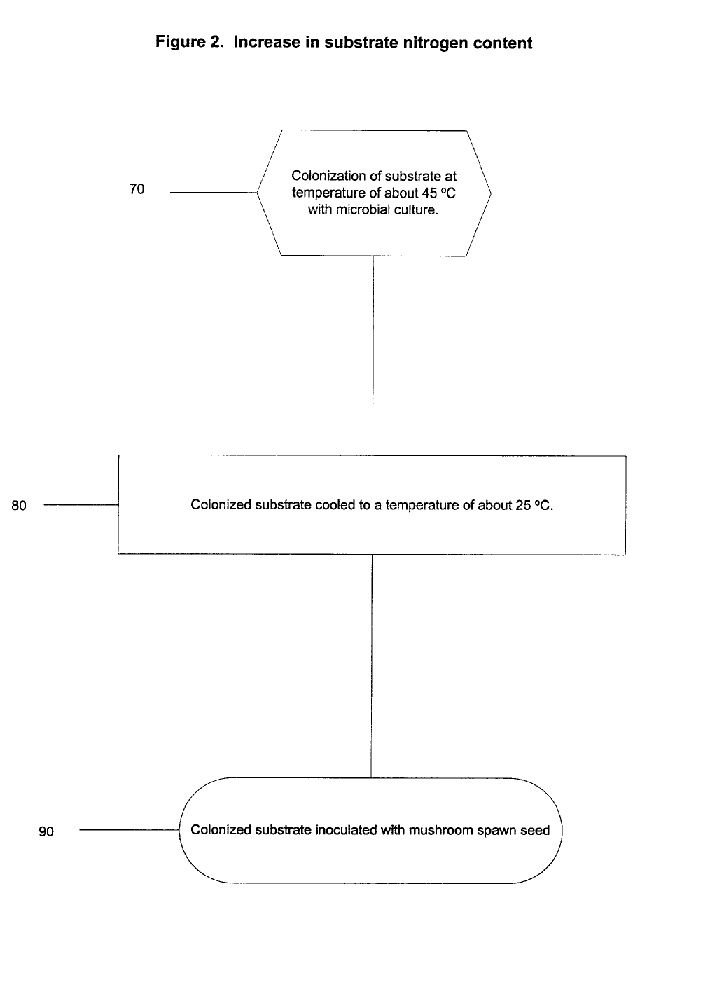

FIG. 2 illustrates increasing the mircrobial nitrogen content of substrate via the colonization of exogenously-derived microbial culture. At least one microbial culture is added to the substrate so as to colonize the substrate, 70. It is preferred that the microbial culture is Scytalidium thermophilum, although a variety of microbial cultures may be used. Colonization with Scytalidium thermophilum is believed to result in the further degradation of the substrate materials and conversion of proteinaceous nitrogen into more available mircrobial nitrogen. It is to be understood that FIG. 2 can be viewed as a continuation of FIG. 1. For example, it is preferred that the substrate be colonized with microbial culture (i.e., 70 of FIG. 2) almost immediately after the cooling of the substrate to about 45° C. (i.e., 60 of FIG. 1). It is preferred that the substrate be colonized with microbial culture within about 2 to 4 days. Preferably the colonized substrate has a pH of between about 6 and 8. The colonized substrate is then cooled to about 25° C., 80. The substrate is then inoculated with mushroom spawn “seed”, 90. It is preferred that the substrate inoculated with mushroom spawn seed is allowed to incubate for about 14 days. Other inoculants, including those listed above, are also highly useful.

The methods of the present invention provide for other significant advantages in substrate production. In particular, the present invention can significantly improve the quantity of substrate produced from organic materials. For example, it has been demonstrated that 1 ton of wheat straw can result in 3 tons of substrate suitable for mushroom growth via the methods of the present invention. This results in less CO₂ evolution, less NH3 evolution and the production of less volatile sulfur compounds. In contrast, traditional composting methods generally produce 2 tons of suitable substrate from 1 ton of wheat straw. Moreover, as described above, the present invention can also provide substrate more suitable for mushroom growth as a results of increase in nitrogen content.

The substrates of the present invention can be formulated for use in mushroom growth. Those skilled in the art will understand that the substrate of the present invention can be formulated to provide a nutrient suitable for mushroom growth. The substrate of the present invention can be formulated with supplements according to U.S. Pat. Nos. 4,764,199 and U.S. Ser. No. 09/506,759, the disclosure of which is hereby incorporated by reference in their entirety. The substrate of the present invention can be formulated with supplements coated with a hydrophilic compound containing a microbe-inhibiting agent as described in U.S. Pat. No. 4,990,173, the disclosure of which is hereby incorporated by reference in their entirety. In addition, the substrate of the present invention can be formulated with mushroom preparations for the control of dipteran pests in accordance with U.S. Ser. No. 09/580,673, the disclosures of which are hereby incorporated by reference in their entirety. It is to be understood that the nitrogen content of the substrate may be reduced when measured relative to the nutrient. For example, when formulated, the nitrogen content of the substrate can be at least about 1.8% based upon the dry weight of nutrient. The nitrogen content of the substrate can also be about 1.8 to 2.0% based upon the dry weight of nutrient.

### EXAMPLES

The invention is further demonstrated in the following examples, which are for purposes of illustration, and are not intended to limit the scope of the present invention.

### Example 1:

Production of Scytalidium Cultures

A number of substrates may be employed for growing Scytalidium cultures for use in this invention. Exemplary among these are:

| Formula #1 | |
|---|---|
| Pre-mix | 18.40% |
| Red's Clay | 18.40% |
| Wheat Middlings | 12.70% |
| Chalk | 1.30% |
| Gypsum | 0.30% |
| Charcoal | 0.20% |
| Water | 48.50% |
| Formula #2 | |
| Rye | 42.00% |
| Chalk | 0.60% |
| Water | 57.40% |
| Formula #3 | |
| Pre-Mix | 20.51% |
| Perlite | 9.28% |
| Wheat Middlings | 14.15% |
| Chalk | 1.45% |
| Gypsum | 0.33% |
| Charcoal | 0.22% |
| Water | 54.05% |
| Formula #4 | |
| Pre-Mix | 43.80% |
| Wheat Bran | 12.50% |
| Chalk | 3.20% |
| Gypsum | 0.80% |
| Charcoal | 0.60% |
| Water | 39.10% |

The substrates are sterilized and inoculated with a pure strain of Scytilidium thermophilium.

The inoculated mass is transferred to bags and maintained under controlled temperature and humidity in a manner identical to that commonly used for production of Agaricus bisporus spawn. It is preferred to proceed until at least about 1×10⁵ CFU per gram of Scytalidium is present.

### Example 2:

Scytalidium Supplemented Compost Versus Compost Supplemented with Biomass of Phase II

Locally harvested, wheat straw having a nitrogen content of approximately 0.5% and a moisture content of<15% was loosened by hand. The straw was loaded by hand with a pitchfork on to a conveyer belt and fed into a cylindrical feed mixer having a volume of 8 m³. Straw and water were added until about 77⅔% of the mixer was filled. 200 grams of an enzyme preparation consisting of cellulase and hemicellulase on a dextzin carrier (Penwest Foods Express) was then added to the mixer.

The contents of the mixer was loaded by hand with a pitchfork into two steel bins after mixing for 20 minutes. The two steel bins each measured 1.55 m high, 1.35 m wide and 1.08 m long. The straw mixture was filled to a height of 80 cm in each bin. The straw mixture content of the two bins had a weight of approximately 625 kg and a moisture content of approximately 75-80%. The two bins were placed in a tunnel measuring 5.59 m long, 1.52 m wide and 3.18 m high and having insulated walls 8 cm thick. The bins thus formed a plenum with a height of 25 cm, the opening in the floor of the tunnel being about 45% of the total floor surface area. The tunnel was fitted with three additional bins for a total of five bins, and the three bins not containing straw closed on the topside with a steel plate.

Process conditions in the tunnel were controlled via a computer having the ability to monitor and regulate fresh air, O_{2,} temperature and air velocity. The contents of the tunnel were heated by steam to a temperature of 45° C. The temperature was maintained between 45° C. and 47° C. for 45 hours. At the conclusion of this high temperature phase, the contents of each bin had a height of 75 cm. The combined weight of the contents of the two bins was approximately 530 kg and the moisture content was 78%.

The contents of the bins were emptied into the mixer, and 70 kg of meal mixture added. The meal mixture included 30% extracted soybean meal, 30% lucerne meal, 20% extracted rapeseed meal and 20% chalk. In addition, 15 kg lime, 10 kg peat moss and 120 liters of water were added. The combined contents were mixed for 20 minutes and then reloaded into the two bins. The combined weight of the contents of the two bins was about 740-750 kg and the moisture content was 75-77%. The filling height of each bin was about 85 cm. The temperature in the tunnel was raised to 100° C. over a period of 2.5 hours using steam. The temperature was then maintained for 2 hours. The temperature was then reduced to 48° C. by cooling with ambient air. Once cooled, the contents of the bins (˜800 kg) were emptied by hand onto a clean, concrete floor.

One-half of the mixture was inoculated by hand at a rate of 0.5-5.0 kg Scytalidium per ton of the mixture and placed into a bin (Bin A). The weight at filling was approximately 370 kg filled to a height of 80 cm. The remaining one-half of the mixture was placed in another bin (Bin B) and inoculated by hand with 8% Phase II compost. The weight at filling was 400 kg and the filling height 80 cm. The bins were placed in the tunnel and the temperature maintained at 47° C. for 72 hours. The mixture in Bin A was then measured and weighed to have a filling height of 75 cm, a weight of 236 kg and a moisture content of 73.5%. The mixture in Bin B was measured and weighed to have a filling height of 75 cm, a weight of 242 kg and a moisture content of 73.8%. The efficiency on a weight basis was 63.4% for Bin A and 60.4% for Bin B. Unlike conventional composting procedure, no smell of ammonia was detected during this process.

Both Bin A and Bin B were then removed from the tunnel. The substrate in both Bin A and Bin B were spawned by hand on the clean concrete floor with strain S100 Agaricus spawn (Sylvan America, Inc.). Both bins were then returned to the tunnel. The mixture of each bin was maintained at a temperature of 25° C. for 14 days.

The colonized substrate was then loaded into growing beds of 1.6 m² with about 80 kg compost/m² and a temperature of 25° C. maintained for 15 days. The growing beds were “cased” at an average depth of 4 cm immediately after filling. The first flush of mushrooms was picked after 14 days. The second and third flushed were picked respectively 8 and 16 days after the first flush. Mushroom yields for each flush and trial are depicted in Table 2.

TABLE 2
Trial Yields (kg/m²)

| | | Substrate with Phase II |
|---|---|---|
| | Substrate with Scytalidium | Biomass |
| 1st flush | 10.35 | 6.68 |
| 2nd flush | 13.90 | 8.40 |
| 3rd flush | 3.45 | 7.55 |
| Total | 27.70 | 22.63 |

Mushroom weights for each trial are depicted in Table 3.

TABLE 3
Mushroom Weight

| | | Substrate with Phase II |
|---|---|---|
| | Substrate with Scytalidium | Biomass |
| Kg/ton substrate | 346.25 | 282.87 |
| Kg/ton DM | 1131.90 | 935.11 |

Mushrooms were weighed after cutting off the basil portion of the stem. Each of the patents, patent applications and publications described herein are hereby incorporated by reference in their entirety. Various modifications of the invention, in addition to those described herein, will be apparent to one skilled in the art in view of the foregoing description. Such modifications are also intended to fall within the scope of the appended claims.

What is claimed is:

1. A mushroom nutrient comprising substrate colonized with an exogenously-derived microbial culture or enzyme, said culture or enzyme being permitted to operate upon said substrate for a period of time effective to digest said substrate.

2. The nutrient of claim 1 wherein said substrate comprises wheat straw, hay, soybean meal, cottonseed meal, gypsum, chicken manure, horse manure, barley straw, oat straw, sugar cane bagasse, lime, urea, alfalfa, lucerne meal, rapeseed meal, or a mixture thereof.

3. The nutrient of claim 1 wherein said microbial culture is a culture of Scytalidium, Micrococcus, Bacillus, Pseudomonas, Klebsiella, Actinomyces, Humicola, Thermoactinomyces, Micromonospora, Nocardia, Chaetomium, Melanocarpus, Paecilomyces, Thermoascus, Rhizomucor, or mixtures thereof.

4. The nutrient of claim 1 wherein the microbial culture is added at a rate of 0.5-5.0 kg per ton of compost, said culture having at least about 1×10⁵ CFU per gram of prepared culture.

5. The nutrient of claim 1 wherein said mircrobial culture is a culture of Scytalidium thermophilium.

6. The nutrient of claim 1 wherein said enzyme is cellulase, hemicellulase proteinase, lipase or laccase.

7. The nutrient of claim 1 wherein said substrate has a moisture content of about 55 to 75%.

8. The nutrient of claim 1 wherein the pH of said substrate is about 6.5 to 8.5.

9. The nutrient of claim 1, wherein said substrate is colonized by a second exogenously-derived microbial culture or enzyme, said second culture or enzyme being permitted to operate upon said substrate for a period of time effective to increase the microbial nitrogen content available to mushrooms of said substrate.

10. The nutrient of claim 9 wherein said second microbial culture is a culture of Scytalidium, Micrococcus, Bacillus, Pseudomonas, Klebsiella, Actinomyces, Humicola, Thermoactinomyces, Micromonospora, Nocardia, Chaetomium, Melanocarpus, Paecilomyces, Thermoascus, Rhizomucor, or mixtures thereof.

11. The nutrient of claim 9 wherein said increase is at least about 25% based upon the mircrobial nitrogen content of the uncolonized substrate.

12. The nutrient of claim 9 wherein said increase is at least about 50% based upon the microbial nitrogen content of the uncolonized substrate.

13. The nutrient of claim 9 wherein said nitrogen content is at least about 2.0% based upon the dry weight of said substrate.

14. The nutrient of claim 9 wherein said nitrogen content is about 2.0 to 2.5% based upon the dry weight of said substrate.

15. The nutrient of claim 9 wherein said nitrogen content is at least about 1.8% based upon the dry weight of said nutrient.

16. The nutrient of claim 9 wherein said nitrogen content is about 1.8 to 2.0% based upon the dry weight of said nutrient.

17. The nutrient of claim 9 wherein the pH of said substrate is about 6 to 8.

18. A mushroom nutrient comprising substrate colonized by exogenously-derived microbial culture, said culture having been permitted to operate upon said substrate for a period of time effective to increase the microbial nitrogen content of said substrate.

19. The nutrient of claim 18 wherein said substrate comprises wheat straw, hay, soybean meal, cottonseed meal, gypsum, horse manure, chicken manure, lucerne meal, rapeseed meal, barley straw, oat straw or a mixture thereof.

20. The nutrient of claim 18 wherein said microbial culture is a culture of Scytalidium, Micrococcus, Bacillus, Pseudomonas, Klebsiella, Actinomyces, Humicola, Thermoactinomyces, Micromonospora, Nocardia, Chaetomium, Melanocarpus, Paecilomyces, Thermoascus, Rhizomucor or mixtures thereof.

21. The nutrient of claim 18 wherein the microbial culture is added at a rate of 0.5-5.0 kg per ton of compost, said culture having at least about 1×10⁵ CFU per gram of prepared culture.

22. The nutrient of claim 18 wherein said increase is at least about 25% based upon the nitrogen content of the uncolonized substrate.

23. The nutrient of claim 18 wherein said increase is at least about 50% based upon the nitrogen content of the uncolonized substrate.

24. The nutrient of claim 18 wherein said nitrogen is at least about 2.0% based upon the dry weight of said substrate.

25. The nutrient of claim 18 wherein said nitrogen content is about 2.0 to 2.5% based upon the dry weight of said substrate.

26. The nutrient of claim 18 wherein said nitrogen content is at least about 1.8% based upon the dry weight of said nutrient.

27. The nutrient of claim 18 wherein said nitrogen content is about 1.8 to 2.0% based upon the dry weight of said nutrient.

28. The nutrient of claim 18 wherein the pH of said substrate is about 6 to 8.

29. A mushroom nutrient comprising mushroom substrate colonized by exogenously-derived microbial culture or enzyme, said substrate having an increased nitrogen content available to the mushroom based upon the nitrogen content of the uncolonized substrate.

30. The nutrient of claim 29 wherein said increase is at least about 25% based upon the nitrogen content of the uncolonized substrate.

31. The nutrient of claim 29 wherein said increase is about 50% based upon the nitrogen content of the uncolonized substrate.

32. The nutrient of claim 29 wherein said nitrogen content is at least about 2.0% based upon the dry weight of substrate.

33. The nutrient of claim 29 wherein said nitrogen content is about 2.0 to 2.5% based upon the dry weight of substrate.

34. The nutrient of claim 29 wherein said nitrogen content is at least about 1.8% based upon the dry weight of nutrient.

35. The nutrient of claim 29 wherein said nitrogen content is about 1.8 to 2.0% based upon the dry weight of nutrient.

36. The nutrient of claim 29 wherein said microbial culture is a culture of Bacillus, Pseudomonas spp or Thermoactinomycetes.

37. The nutrient of claim 29 wherein said microbial culture is a culture of Scytalidium thermophilium or Micrococcus spp.

38. The nutrient of claim 29 wherein said enzyme is cellulase, hemicellulase proteinase, lipase or laccase.

39. The nutrient of claim 29 wherein the microbial culture has at least about 1×10⁵ CFU per gram of prepared culture.

40. A method of preparing mushroom nutrient comprising colonizing substrate with an exogenously-derived microbial culture or enzyme so as to digest said substrate.

41. The method of claim 40 further comprising maintaining a temperature of about 35 to 75° C.

42. The method of claim 40 further comprising maintaining said substrate at a pH of about 7 to 8.

43. The method of claim 40 further comprising continuously increasing the moisture content of said colonized substrate.

44. The method of claim 40 further comprising maintaining the moisture content of said colonized substrate at about 55 to 70%.

45. The method of claim 40 further comprising heating said colonized substrate for a period of time sufficient to pasteurize or sterilize said substrate.

46. The method of claim 40 further comprising pasteurizing or sterilizing said colonized substrate by heating at a temperature of about 84° C. for about 12 hours.

47. The method of claim 40 further comprising pasteurizing or sterilizing said colonized substrate by heating at a temperature of about 90° C. for about 6 hours.

48. The method of claim 40 further comprising pasteurizing or sterilizing said colonized substrate by heating at a temperature of about 100° C. for about 2 hours.

49. The method of claim 40 further comprising pasteurizing or sterilizing said colonized substrate by heating at a temperature of about 170° C. for about 10 minutes.

50. The method of claim 40 further comprising cooling said pasteurized or sterilized substrate to a temperature of about 45° C.

51. The method of claim 40 further comprising colonizing said substrate with a second exogenously-derived microbial culture so as to increase the nitrogen content of said colonized substrate based upon the nitrogen content of uncolonized substrate.

52. The method of claim 51 wherein said increase is at least about 25% based upon the nitrogen content of the uncolonized substrate.

53. The method of claim 51 wherein said increase is at least about 50% based upon the nitrogen content of the uncolonized substrate.

54. The method of claim 51 wherein said nitrogen content is at least about 2.0% based on the dry weight of substrate.

55. The method of claim 51 wherein said nitrogen content is about 2.0 to 2.5% based on the dry weight of substrate.

56. The method of claim 51 wherein said nitrogen content is about 1.8% based upon the dry weight of nutrient.

57. The method of claim 51 wherein said nitrogen content is about 1.8 to 2.0% based upon the dry weight of nutrient.

58. The method of claim 51 further comprising maintaining the substrate at a pH of about 6 to 8.

59. A mushroom growth nutrient prepared by the method of claim 40.

60. A mushroom growth nutrient prepared by the method of claim 51.

61. A method of growing mushrooms comprising inoculating said substrate of claim 51 with mushroom spawn.

62. A mushroom growth nutrient prepared by the method of claim 18.

63. A mushroom growth nutrient prepared by the method of claim 29.
